(19) United States
(12) Patent Application Publication
(10) Pub. No.: US 2007/0142888 A1
(43) Pub. Date: Jun. 21, 2007

(54) Implantable leads and methods of using the same

(76) Inventor: Alfonso Chavez, San Jose, CA (US)

Correspondence Address: PEPPER HAMILTON LLP, ONE MELLON CENTER, 50TH FLOOR, 500 GRANT STREET, PITTSBURGH, PA, 15219, US

(21) Appl. No.: 11/312,184

(22) Filed: Dec. 20, 2005

(51) Int. Cl. A61N 1/05

(52) U.S. Cl. 607/116

(57) ABSTRACT

An implantable medical lead having an indicator band is disclosed. Implanting the lead is simplified and accurate localization of lead electrodes is improved by using an indicator band to aid in aligning the lead within a receiving port of an implantable lead connector assembly. The medical lead may be used for neurostimulation.

### TECHNICAL FIELD

The disclosed embodiments relate to implantable leads, and more particularly to implantable medical leads that exhibit indicator bands for neurological application.

### BACKGROUND

In medical diagnosis and treatment of various brain disorders, including epilepsy, Parkinson's disease, sleep disorders and psychiatric ailments, it is customary and frequently useful to analyze electrical signals originating in the brain. It is also becoming accepted to apply electrical stimulation to various structures of the brain for both diagnostic and therapeutic purposes. Furthermore, chronic electrical stimulation is contemplated as a direct treatment for various disorders. For example, an implantable neurostimulator that is coupled to an implantable medical lead having one or more deep brain electrodes or depth electrodes is described in U.S. Pat. No. 6,016,449 to Fischell et al. (hereinafter “Fischell”).

Generally, external equipment, such as a cranially implantable neurostimulator device, is connected to acutely implanted brain electrodes located on an implantable medical lead. Typical implantable medical leads are flexible small-diameter (usually 1-1.5 mm) round leads having one or more distal electrodes located coaxially along the distal end of the lead. It is also possible to employ an implantable medical lead having scalp electrodes instead of depth electrodes for certain types of electroencephalogram monitoring and analysis along the scalp; however, precise localization of the electrode is less feasible with scalp electrodes.

Generally, implantation of an implantable medical lead involves surgically exposing an insertion site in the cranium that is an operative distance from an electrode site. An electrode site may be chosen at any appropriate stage of the surgical procedure, including pre-surgically in an operative planning stage; intraoperatively after a craniotomy has been performed or a burr hole has been made; or intraoperatively after one or more other procedures, such as functional mapping, have been performed. After an insertion site is created, the lead is then inserted to the desired electrode site along a predetermined direction to a predetermined depth.

Common requirements for implantable medical leads include flexibility, strength, and durability. The extent of such qualities is dependent upon the nature of the use, for example, temporary or permanent implantation. While material selection and certain construction techniques can be tailored to assist in meeting these prescribed characteristics, increasing the ease with which a delicate lead is implanted is also generally considered.

Systems for electrically monitoring and stimulating the brain are increasingly important in the medical diagnosis and treatment of various brain disorders. Therapeutic neurostimulatory devices typically include one or more leads having at least one electrode operatively situated in the brain or other neural tissue and linked to a signal processor for detecting neurological activity and to a pulse generator for providing electrical stimuli.

Many functional and aesthetic advantages can be achieved by implanting the signal processing and pulse generator portions of neurostimulator devices in the cranium. From a purely esthetic point of view, the leads need not run along the scalp and down the neck. Connecting implanted electrodes to devices located in other regions of the body mandates that the leads be lengthy. For instance, the Medtronic Activa® device uses leads that are tunneled along the neck and down the chest to the pectoral region where the neurostimulator resides. Such a pathway subjects the leads to increased risk of fatigue and to a higher susceptibility to noise from a variety of external electromagnetic sources.

Fischell describes a system that can be implanted beneath the scalp. Fischell discloses a responsive detection and stimulation system for the early recognition and prompt treatment of a neurological event arising from neurological disorders, such as epilepsy, migraine headaches and Parkinson's disease. In Fischell's device, the entire implantable portion of the system for treating neurological disorders lies beneath the patient's scalp. By placing the entire system within the cranium, as opposed to extending wires into or through the neck to a control module in the chest, the probability of wire malfunction due to repeated wire bending is drastically reduced.

Other exemplary devices implanted in the cranium for applying electrical stimulation therapies to electrodes situated at appropriate locations include cochlear implants. By implanting an electrode lead connection device into a patient's cranium, a reliable electrical connection between the electrodes and an implantable neurostimulatory and/or monitoring device can be ensured. In such devices, an interposer that is secured inside a connector housing and is electrically connected to the device can be used.

One problem with leads used with conventional systems is that it is difficult to properly place or position the leads in the appropriate receptacle or receiving port. An implantable electrode lead that is easier to place within implantable lead connector assemblies or similar devices would be desirable.

Another problem with conventional leads is that even when an implantable electrode lead is appropriately placed within a receptacle or receiving port, the lead may move prior to fixation within a conventional system. For example, if the conventional system includes two halves that are joined together to affix the lead, moving the system prior to joining the two halves, but after placing the lead may result in the lead being moved from its intended position.

### SUMMARY

In an embodiment, a lead may include a body having a proximal end and a distal end, an electrode at the distal end, a terminal at the proximal end, and an indicator band proximal of the electrode. The indicator band may provide visual verification of the correct placement of the lead in a receiving port. The indicator band may include one or more of polyimide, platinum, PEEK, a biocompatible polymer, and a radio-opaque material detectable by an X-ray. The indicator band may be visible to the unaided eye, such as at a distance of about 12 inches or a distance of about 18 inches. The indicator band may be about 0.015 inches in length. The indicator band may include a coating, such as silicone. The indicator band may be located between about 0.642 inches and about 0.652 inches from the proximal end of the lead. The indicator band may be on the body. The indicator band may be embedded within the body. The indicator band may not protrude from the body. The lead may include a substantially longitudinal conductor. The indicator band may be disposed around the conductor. The body may be one or more of translucent and biocompatible. The body may include an elastomer. The body may include silicone.

In an embodiment, a lead for use with a medical device may include a body having a proximal end adapted to be inserted into a connector assembly and a distal end, and an indicator band adjacent to the proximal end and positioned to substantially coincide with an edge of the connector assembly when the lead is properly inserted into the connector assembly. The lead may include one or more of an electrode lead, a catheter, a fiber optic device, a thermal device and a longitudinal lumen.

In an embodiment, an implantable lead connector assembly and electrode lead combination may include an implantable lead connector assembly containing an interposer, and an electrode lead insertable into the interposer. The electrode lead may have an indicator band thereon. The indicator band may coincide with an edge of the implantable lead connector assembly when the electrode lead is properly inserted into the implantable lead connector assembly. The implantable lead connector assembly may include one or more electrically conductive members. The electrode lead may include one or more electrodes. If the electrode lead is inserted into the implantable lead connector assembly such that the distance between the edge of the implantable lead connector assembly and the indicator band is less than or equal to the width of the indicator band, each electrode may contact a corresponding electrically conductive member.

In an embodiment, a method of placing a lead in a receiving port may include positioning a lead having an indicator band into a receiving port, and affixing the lead into the receiving port. The indicator band may provide visual verification of correct placement in the receiving port. The indicator band may be located such that the indicator band aligns with an entry point of the receiving port when the lead is correctly placed in the receiving port. The indicator band may be located such that if a distance between an entry point of the receiving port and the indicator band is less than or equal to the width of the indicator band, the lead is correctly placed in the receiving port. The indicator band may include one or more of platinum, polyimide, PEEK, a biocompatible polymer and a radio-opaque material. The indicator band may be visible to the unaided eye.

### BRIEF DESCRIPTION OF THE DRAWINGS

FIG. 1 depicts an exemplary electrode lead having an indicator band according to an embodiment.

FIG. 2 depicts an exemplary coiled electrode lead having an indicator band according to an embodiment.

FIGS. 3A and 3B depict exemplary coiled electrode leads having an indicator band inserted in an interposer according to an embodiment.

FIG. 4 depicts an exemplary disassembled electrode lead connector having a clamp housing and a connector carriage with a split interposer seated therein and an exemplary electrode lead according to an embodiment.

FIG. 5A is a top perspective view of an exemplary interposer for holding fuzz button contacts according to an embodiment.

FIG. 5B is a bottom perspective view of the interposer of FIG. 5A according to an embodiment.

FIG. 6 depicts an exemplary assembled lead connector containing a single electrode lead according to an embodiment.

### DETAILED DESCRIPTION

Before the present methods, systems and materials are described, it is to be understood that this disclosure is not limited to the particular methodologies, systems and materials described, as these may vary. It is also to be understood that the terminology used in the description is for the purpose of describing the particular versions or embodiments only, and is not intended to limit the scope.

It must also be noted that as used herein and in the appended claims, the singular forms “a,” “an,” and “the” include plural references unless the context clearly dictates otherwise. Thus, for example, reference to a “band” is a reference to one or more bands and equivalents thereof known to those skilled in the art, and so forth. Unless defined otherwise, all technical and scientific terms used herein have the same meanings as commonly understood by one of ordinary skill in the art. Although any methods, materials, and devices similar or equivalent to those described herein can be used in the practice or testing of embodiments, the preferred methods, materials, and devices are now described. All publications mentioned herein are incorporated by reference in their entireties. Nothing herein is to be construed as an admission that the embodiments described herein are not entitled to antedate such disclosure by virtue of prior invention.

As used herein, the term “about” means plus or minus 10% of the numerical value of the number with which it is being used. Therefore, about 50% means in the range of 45%-55%.

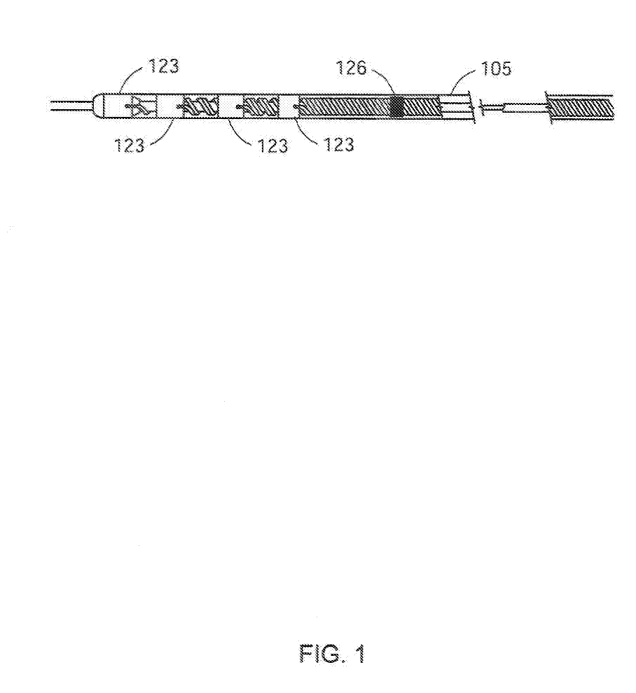

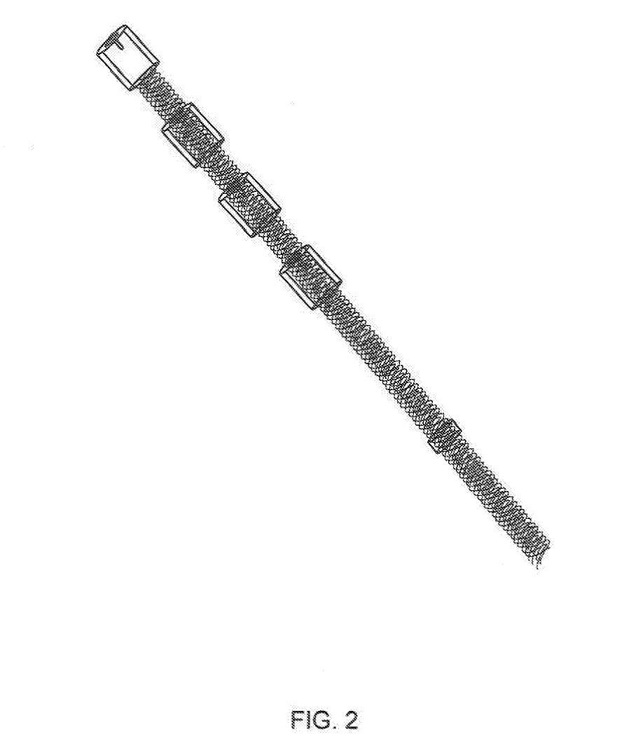

FIG. 1 depicts an exemplary electrode lead having an indicator band according to an embodiment. As shown in FIG. 1, a lead may include a body 105 having a proximal end and a distal end, at least one electrode 123 at the distal end, at least one terminal at the proximal end, and at least one indicator band 126. In an embodiment, the body 105 may be translucent. In an embodiment, the body 105 may be biocompatible. In an embodiment, the body 105 may include an elastomer. In an embodiment, the body 105 may include silicone. The at least one band 126 may be proximate to the at least one electrode 123. In an embodiment, the at least one indicator band 126 may be positioned on the body 105. In an embodiment, the indicator band 126 may be embedded within the body 105. In an embodiment, the indicator band 126 may not protrude from the body 105. In an embodiment, the indicator band 126 may be positioned such that visual verification of the correct placement of the electrode lead in a receiving port of a connector assembly may be performed. In an embodiment, the lead may include one or more substantially longitudinal conductors (not shown). In an embodiment, the indicator band 126 may be disposed around the conductors. An internal view showing an exemplary coiled electrode lead is depicted in FIG. 2.

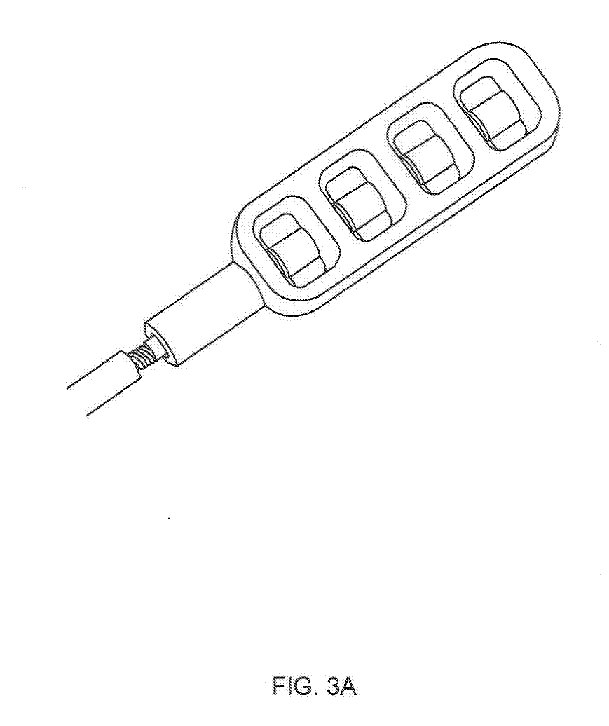

FIGS. 3A and 3B depict exemplary coiled electrode leads having an indicator band inserted in an interposer according to an embodiment. In an embodiment, an implantable lead connector assembly and electrode lead combination may include an implantable lead connector assembly containing an interposer and an electrode lead insertable into the interposer. The electrode may have at least one indicator band thereon. The at least one band may coincide with an edge of the implantable lead connector assembly if the electrode lead is properly inserted into the implantable lead connector assembly, as shown in FIG. 3B.

In an embodiment, a method of placing an electrode lead in a receiving port may include positioning an electrode lead having at least one indicator band into a receiving port to provide visual verification of correct placement in the receiving port. The indicator band may be located on the electrode lead such that the indicator band aligns with, for example, the entry point of the receiving port when the electrode lead is correctly placed in the receiving port. The electrode lead may then be affixed within the receiving port, and its position may be verified.

The indicator band may comprise one or more of a variety of suitable materials, such as polyimide, platinum, PEEK, and/or any other biocompatible polymer. Polyimide material may be preferred for the indicator band due to its natural coloring.

In an embodiment, the indicator band may also include a radio-opaque doping agent or be made of a radio-opaque material so that the band may be visible within an X-ray.

In an embodiment, the indicator band may be visible to the unaided eye. In an embodiment, the indicator band may be visible to the unaided eye at a distance of about 12 inches. In an embodiment, the indicator band may be visible to the unaided eye at a distance of about 18 inches.

The indicator band may not interfere with the implantable electrode lead. For example, in an embodiment a coiled implantable electrode lead may be bent up to a radius of about 0.2 inches. In order to prevent damage to the lead, the gap between the indicator band and the casing of the lead should be less than or equal to the length of the indicator band. Accordingly, in such an embodiment, the length of the indicator band may be less than about 0.049 inches (as determined by performing a tolerance analysis measurement) in order to prevent interference when the lead is bent to a radius of about 0.2 inches. In an embodiment, the indicator band may be about 0.015 inches in length. In an embodiment, the length of the indicator band may be chosen to assist in the indication of satisfactory seating within a receiving port given a worst-case seating tolerance within the receiving port and/or worst-case tolerances for manufacture of the lead.

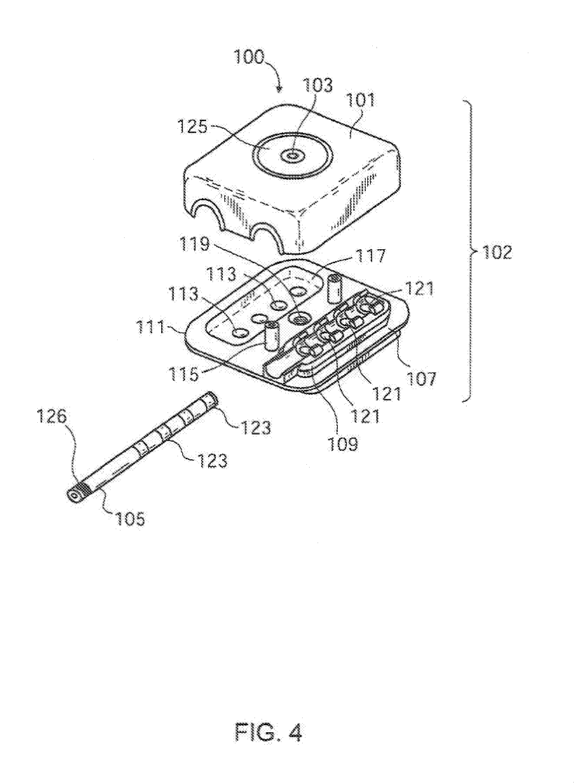

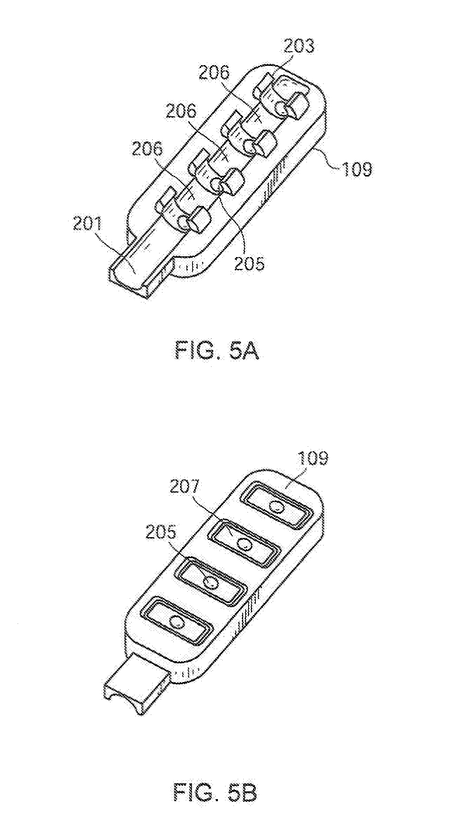

In an embodiment, the placement of the indicator band on the lead may be derived from the depth of a connector into which the lead is inserted. Moreover, tolerances for each of the electrode placements may be considered in determining the placement of the indicator band. In other words, even in a worst-case electrode placement scenario (as far as tolerances for electrode placement are concerned), the indicator band, when properly aligned with the entry point of a connector assembly may still indicate that the electrodes properly contact electrically conductive members in a connector assembly, such as those shown in FIGS. 5A and 5B. Accordingly, if a lead is completely inserted into a connector assembly, a failed connection may not occur. Preferably, if a lead is inserted into a connector assembly such that a visible gap between the indicator band and the connector assembly is less than or equal to the width of the indicator band, a proper connection between each electrode of the lead and each corresponding electrically conductive member of the connector assembly may result.

The indicator band may be placed on the implantable lead in a fixed position, such that alignment of the indicator band with a particular structure of a device indicates correct placement of the electrode lead within the device. For example, correct lead placement may be indicated by the position of the indicator band with respect to an entry of a receiving port of the device.

In an embodiment, the implantable electrode lead may include a fixed indicator band. The indicator band may be fixed such that the proximal edge of the band is about 0.647±0.005 inches from the proximal end of the lead. In an embodiment, the length of the indicator band may be about 0.015 inches.

The indicator band may be encapsulated or coated. Encapsulation or coating may provide electrical performance and may prevent stress to the lead at the location of the indicator band on the lead. In an embodiment, a suitable material for encapsulating or coating the indicator band may include silicone and/or any other material known in the art that is suitable for implantation.

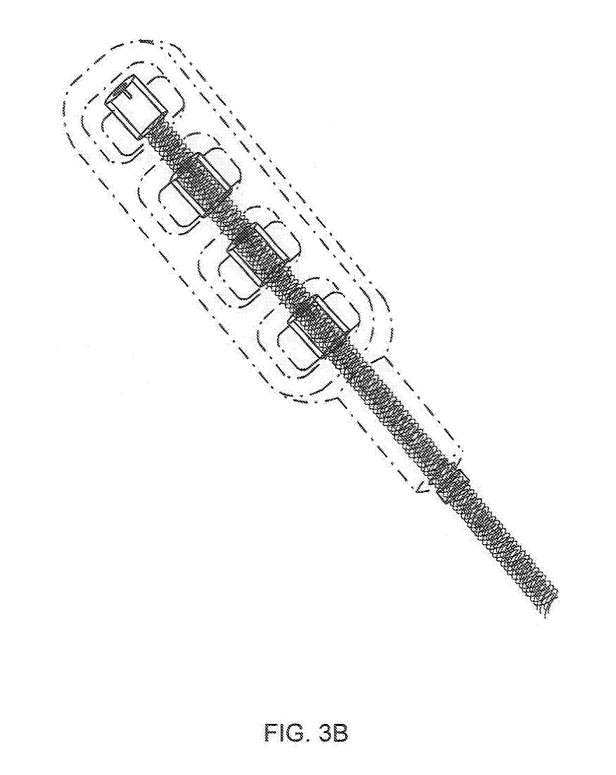

FIG. 4 illustrates an implantable lead connector assembly 100 connectable to an exemplary electrode lead 105 having indicator bands 126. The connector housing 102 of the lead connector assembly 100 may include, for example, three parts: a clamp housing 101, a connector carriage 107 and an interposer or removable seal 109. The lead connector assembly 100 may include: a) a functional clamp that holds the lead connector assembly closed and simultaneously holds the various electrode leads in place while isolating the various electrical contacts, b) an interposer or seal that accepts the proximal end of the various electrode leads and cooperatively (upon clamping or closing the inventive connector assembly) seals the various electrical contacts to form a circuit with the lead contacts such that the information or stimulus passing through the connector is isolated into the circuitry as intended by the designer, and c) a connector carriage supporting the interposer and serving as a passageway for electrical signals into and out of the attached stimulator or signal processor. The connector carriage may assist in performing the clamping function.

As shown in FIG. 4, the electrode lead 105 having at least one indicator band 126 may be received by, held in place by, and/or positioned by an interposer or removable seal 109 that accommodates and electrically isolates electrically conductive members 121 between the clamp housing 101 and the connector carriage 107. As used herein, the terms “interposer,” “removable seal” and “interposer seal” may be used to describe the component designated “109” in FIG. 4 because of the multiple functions performed by that component.

Each of the electrically conductive members 121 may contact a corresponding lead terminus or proximal contact 123 on electrode lead 105 having indicator bands 126. A user may verify the connection by visually inspecting the indicator bands 126. This connection may form an electrical circuit between the electrically conductive members 121 and the electrode lead 105. In an embodiment, fluid, such as a conductive fluid, may be present within the confines of the connector assembly 102 when the device is closed and operable. The interposer 109 may seal one electrically conductive member 121 from all other electrically conductive members. As such, the interposer 109 may tend to eliminate erroneous information from being passed to the attached signal processor and may certify the passage of stimulation to appropriate sectors of the brain. Such a lead connector assembly 100 may receive one or more electrode leads for connection to an implantable device.

Returning to FIG. 4, the connector housing 102 may include at least a clamp housing 101 and a connector carriage 107. In the embodiment shown in FIG. 4, these two components are depicted to be separable. Such separability may facilitate installation and replacement of an electrode lead 105. In an embodiment, the clamp housing 101 and connector carriage 107 may be integrated into a single element. In an embodiment, a hinge may join the clamp housing 101 and connector carriage 107. The clamp housing 101 may comprise a biocompatible material, such as polyetheretherketone (PEEK). The interior of the clamp housing 101 may hold the interposer 109 in place and conform in shape to that interposer. Because embodiments of an interposer 109 may vary in shape (see below), a variety of clamp housing designs may be contemplated, and the interior shape of the clamp housing 101 may be limited to one having a recessed region that fits the shape of the interposer.

As shown in FIG. 4, the connection to the electrode lead 105 may include, for example, two parts: at least one feed-through pin 113 and a compressible electrical connection member 121. The compressible electrical connection members 121 may include, for example, spring contacts, fuzz button connectors and/or other similarly functional components. In an embodiment, the compressible electrical connection members 121 may include a spring contact. A spring contact may be an open or closed loop of a biocompatible, conductive material, such as a pure metal or an alloy (such as 80-20 Platinum-Iridium), that achieves a predictable amount of opposing force when compressed.

In an embodiment, the compressible electrical connection members 121 may be fuzz buttons. Fuzz buttons may be made from a very fine diameter wire, for example, of Pt—Ir. The wire may be formed into a shape approximating a cylinder. Such forms are commercially available from Tecknit Co of Cranford, N.J. Other shapes (for example, multiple coils) and other conductive materials may also serve as compressible electrical connection members.

Each feed-through pin 113 may extend through the baseplate 111, project externally, and attach directly or indirectly, to the implantable device. In an embodiment, each feed-through pin 113 may include a suitable biocompatible, corrosion-resistant, highly conductive metal, such as a member of the Noble Metal group, such as platinum, palladium or iridium or an alloy of, for example, platinum and iridium. The feed-through pin 113 and the compressible electrical connection member 121 may be fabricated from the same conductive material or even made as a single element.

As noted above, the lead connector assembly 100 may accept, for example, two interposer seals 109 each accommodating, for example, four electrical connections to each electrode lead 105. Fewer or more interposers 109 and/or electrical connections 123 may be used within the scope of this disclosure, and embodiments will be apparent to those of ordinary skill in the art. The lead connector assembly 100 may be configured to connect to electrode leads having a much higher density of electrodes by designing the location or spacing of the electrically conductive members and interposer openings to conform to the number and spacing of the various electrode termini 123.

The connector housing 102 may be assembled by aligning the clamp housing 101 and the connector carriage 107. Ancillary assembly design aids such as alignment posts 115 on the connector carriage 107 and matching holes (not shown) in the underside of the clamp housing 101 may assist in aligning the connector carriage to the clamp housing. Such alignment posts may be installed into mating holes or sockets in the connector carriage 107 (or the connector housing 102) or may be formed integrally with the carriage or housing. The clamp housing 101 and connector carriage 107 may be secured together by a fastener 103 once the one or more electrode leads 105 are properly positioned in interposer 109.

The inventive lead connector assembly 100 may be assembled around the electrode leads 105. The proximal end (or “connector end”) of the depicted electrode lead 105 may have a number of proximal electrode contacts or termini 123, such as the ring-type contacts shown in FIG. 4. Each proximal contact 123 may be in electrical contact with the distal lead electrodes implanted into, for example, the brain. The connector end of the electrode lead 105 may fit into the interposer 109. The interposer 109 may be made from any suitable biocompatible insulating material, such as a silicone (for example, MED4950 silicone from NuSil Technology). In an embodiment, the material may be elastomeric. The interposer 109 may include an axial passageway that permit lengthwise entrance of an electrode lead, such as 105, and openings extending generally radially to the axial passageway that may contain the electrical connection members 121 discussed at length above. The physical and electrical contact between each proximal electrode contact 123 of the electrode lead 105 may thus be formed.

FIGS. 5A and 5B depict an exemplary interposer. Other variations of interposers are described in U.S. Pat. No. 6,662,035 entitled “Implantable Lead Connector Assembly for Implantable Devices and Methods of Using It” filed Sep. 13, 2001 and herein incorporated by reference in its entirety.

In FIG. 5A, the interposer 109 may have an axial passageway or channel 201 and a series of bendable, but substantially rigid, clips 203 adapted to fix the position of an electrode lead (for example, 105 in FIG. 4). Such an embodiment may be used, for example, with compressible electrically conductive members 205 that are fuzz buttons. The interposer 109 may hold electrically conductive members 205 in the openings exposed to the electrode contacts of the electrode lead. Many variations of the overall shape of the interposer 109 of FIG. 5A may be used within the scope of this disclosure and will be apparent to those of ordinary skill in the art.

FIG. 5B illustrates the underside of the interposer 109 shown in FIG. 5A. As shown in FIG. 5B, the interposer 109 may contact the baseplate of the connector carriage 107 as shown in FIG. 4. The compressible electrically conductive members 205 may extend through the interposer 109 and may be adapted to electrically contact with the feed-through pins 113, shown in FIG. 4. The compressible electrically conductive members 205 may be held in the openings of the interposer 109 by various structures and/or adhesives.

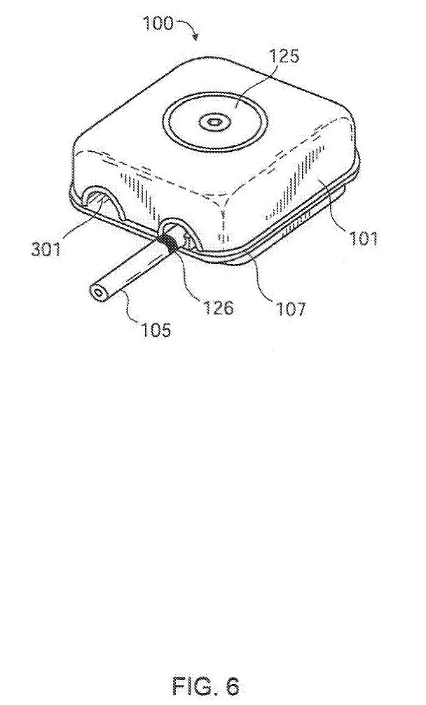

FIG. 6 illustrates the assembled and sealed lead connector assembly 100. After inserting the electrode lead 105 into the interposer 109, the interposer may be held between the clamp housing 101 and connector carriage 107. The fastener 103 may be engaged, locking the clamp housing 101 to the connector carriage 107, and making electrical contact between the electrode contact of the electrode lead 105 and the electrically conductive members 125 (such as the fuzz button connector and the feed-through pin). The fastener 103 may compress the compressible fuzz button connector 125 against the electrode lead 105, and may also seal each contact of the electrode lead within the interposer 109. It is within the scope of this disclosure that the interposer 109 and the complementary section that fits above the portion shown in FIG. 5A within the clamp housing 101 (all discussed above in more detail), may be detachable or removable from the connector assembly 100. In an alternate embodiment, each interposer seal may be fixed (e.g., glued) respectively within clamp housing 101 and the connector carriage 107.

In an embodiment, a lead including at least one indicator band may be used for a different purpose than as an electrode. For example, a lead including an indicator band may be used as a catheter, a fiber optic device, a thermal device (such as a temperature sensor), a lumen (such as a longitudinal lumen) and the like. The term “lead,” as used herein, includes all such devices, similar devices embodying similar principles and other devices that are apparent to those of ordinary skill in the art based upon the disclosure herein, whether for a medical device or any other device, that needs to be inserted to a proper depth into a connector assembly. Such leads need not include terminals and/or electrodes.

It will be appreciated that variations of the above-disclosed and other features and functions, or alternatives thereof, may be desirably combined into many other different systems or applications. It will also be appreciated that various presently unforeseen or unanticipated alternatives, modifications, variations or improvements therein may be subsequently made by those of ordinary skill in the art which are also intended to be encompassed by the following claims.

What is claimed is:

1. A lead, comprising:
a body having a proximal end and a distal end;
an electrode at the distal end;
a terminal at the proximal end; and
an indicator band proximal of the electrode.

2. The lead of claim 1 wherein the indicator band provides visual verification of the correct placement of the lead in a receiving port.

3. The lead of claim 1 wherein the indicator band comprises polyimide.

4. The lead of claim 1 wherein the indicator band comprises platinum.

5. The lead of claim 1 wherein the indicator band comprises PEEK.

6. The lead of claim 1 wherein the indicator band comprises a biocompatible polymer.

7. The lead of claim 1 wherein the indicator band comprises a radio-opaque material detectable by an X-ray.

8. The lead of claim 1 wherein the indicator band is visible to the unaided eye.

9. The lead of claim 1 wherein the indicator band is visible to the unaided eye at a distance of about 12 inches.

10. The lead of claim 1 wherein the indicator band is visible to the unaided eye at a distance of about 18 inches.

11. The lead of claim 1 wherein the indicator band is about 0.015 inches in length.

12. The lead of claim 1 wherein the indicator band comprises a coating.

13. The lead of claim 12 wherein the coating comprises silicone.

14. The lead of claim 1 wherein the indicator band is located between about 0.642 inches and about 0.652 inches from the proximal end of the lead.

15. The lead of claim 1 wherein the indicator band is on the body.

16. The lead of claim 1 wherein the indicator band is embedded within the body.

17. The lead of claim 16 wherein the indicator band does not protrude from the body.

18. The lead of claim 1 wherein the lead comprises a substantially longitudinal conductor.

19. The lead of claim 18 wherein the indicator band is disposed around the conductor.

20. The lead of claim 1 wherein the body is translucent.

21. The lead of claim 1 wherein the body is biocompatible.

22. The lead of claim 1 wherein the body comprises an elastomer.

23. The lead of claim 1 wherein the body comprises silicone.

24. A lead for use with a medical device, the lead comprising:
a body having a proximal end and a distal end, wherein the proximal end is adapted to be inserted into a connector assembly; and
an indicator band adjacent to the proximal end, wherein the indicator band is positioned to substantially coincide with an edge of the connector assembly when the lead is properly inserted into the connector assembly.

25. The lead of claim 24 wherein the lead comprises an electrode lead.

26. The lead of claim 24 wherein the lead comprises a catheter.

27. The lead of claim 24 wherein the lead comprises a fiber optic device.

28. The lead of claim 24 wherein the lead comprises a thermal device.

29. The lead of claim 24 wherein the lead comprises a longitudinal lumen.

30. An implantable lead connector assembly and electrode lead combination, comprising:
an implantable lead connector assembly containing an interposer;
an electrode lead insertable into the interposer, wherein the electrode lead has an indicator band thereon, wherein the indicator band coincides with an edge of the implantable lead connector assembly when the electrode lead is properly inserted into the implantable lead connector assembly.

31. The implantable lead connector assembly and electrode lead combination of claim 30, wherein the implantable lead connector assembly comprises one or more electrically conductive members, wherein the electrode lead comprises one or more electrodes, and wherein if the electrode lead is inserted into the implantable lead connector assembly such that the distance between the edge of the implantable lead connector assembly and the indicator band is less than or equal to the width of the indicator band, each electrode contacts a corresponding electrically conductive member.

32. A method of placing a lead in a receiving port, comprising:
positioning a lead having an indicator band into a receiving port; and
affixing the lead into the receiving port,
wherein the indicator band provides visual verification of correct placement in the receiving port.

33. The method of claim 32 wherein the indicator band is located such that the indicator band aligns with an entry point of the receiving port when the lead is correctly placed in the receiving port.

34. The method of claim 32 wherein the indicator band is located such that if a distance between an entry point of the receiving port and the indicator band is less than or equal to the width of the indicator band, the lead is correctly placed in the receiving port.

35. The method of claim 32 wherein the indicator band comprises platinum.

36. The method of claim 32 wherein the indicator band comprises polyimide.

37. The method of claim 32 wherein the indicator band comprises PEEK.

38. The method of claim 32 wherein the indicator band comprises a biocompatible polymer.

39. The method of claim 32 wherein the indicator band comprises a radio-opaque material.

40. The method of claim 32 wherein the indicator band is visible to the unaided eye.
